(19) United States
(12) Patent Application Publication
(10) Pub. No.: US 2014/0275742 A1
(43) Pub. Date: Sep. 18, 2014

(54) Infant Blanket with Insertable and Programmable Devices to Provide Stimulation and Soothing to an Infant

(71) Applicant: Unger Andrew, Bethlehem, PA (US)

(72) Inventor: Unger Andrew, Bethlehem, PA (US)

(21) Appl. No.: 13/803,417

(22) Filed: Mar. 14, 2013

(51) Int. Cl. A61M 21/02; A61N 5/06

(52) U.S. Cl. CPC A61M 21/02; A61N 5/0618; A61N 5/0621
USPC 600/28; 600/27

(57) ABSTRACT

A stimulation and soothing device for an infant is disclosed. The device includes a pouch large enough to accommodate the lower torso of an infant when the infant is placed in a reposed position therein. A backing piece includes first and second wings that wrap over the infant's shoulder and arms and secure to the pouch front panel. Each wing includes a pocket in which an insertable and programmable device may be housed. The insertable and programmable devices are provided for infant stimulation and soothing and may include a sound transducer and/or a portable digital media player. Additional pockets are provided for a temperature probe and for a phototherapy blanket.

### FIELD OF THE INVENTION

The invention relates generally to a blanket including insertable programmable devices for stimulating and soothing an infant. More specifically, the insertable devices may include a heartbeat simulator and/or a digital media player to provide a soothing effect to the infant as well to provide stimulating audio content to the infant such as a mother's voice, music, etc.

### BACKGROUND OF THE INVENTION

Parent-infant attachment or bonding is crucial for continuous and successful development of a newborn infant, especially in the case of preterm/premature infants (hereinafter “preterm infants”). Research has shown that newborns, including preterm infants, have a uniquely innate form of interaction and attachment with their parents early on in their developmental process. That is, attachment between a newborn infant and the infant's parent, especially the infant's mother, provides essential physical, emotional and psychological needs required for orderly infant development and maturity.

In the past, the importance of an infant's emotional and psychological needs through parent-infant attachment was not fully understood in the medical profession and thus not well documented. Such level of ignorance has since changed, as contemporary child development research has gradually established the importance of a parent's voice, heartbeat, breathing and physical contact, to the early development of a preterm infant or new-born infant. Contemporary research has also established that an infant recognizes his or her birth mother through the mother's scent and is thus capable of noticing the presence or absence of the mother.

The absence of an infant's mother affects the infant's security and often has an adverse effect on the orderly development of the infant. In contrast, when an infant's mother is present, the infant benefits from the soothing tone of the mother's voice and the rhythm of both the mother's heartbeat and breathing. Thus, it is well established that parent-infant attachment is necessary for fulfilling the emotional and psychological needs, and for the orderly development of a newborn infant. Skin-to-skin contact between the mother and the baby are also important for orderly development of a newborn infant. The attachment phenomenon, which promotes emotional, psychological and orderly development of an infant is even more important and pronounced when dealing with premature infants.

An infant's birth places enormous stress and difficulty on parents and supportive relatives. In today's society, mothers are often required to return to work within a relatively short period of time, e.g., six weeks, following the infant's birth. Often the mother is confronted with no choice but to place the infant in day care where the infant spends its crucial early development period lying on its back in a crib. In the case of a pre-term infant, the infant is often required to remain in the hospital after its birth where the infant may receive “kangaroo care” which is a technique practiced on newborn infants, usually preterm, wherein the infant is held skin-to-skin, with an adult, such as the mother or father. However, kangaroo care often must be restricted to a few hours per day, based upon a parent's availability and other factors.

It is not uncommon for a mother to feel extremely helpless and often depressed when repeatedly confronted with challenges and complexity associated with newborns and premature infants. Although advances in technology have played a significant part in the survival rate of premature infants, research has shown that a mother's, voice, heartbeat and breathing contribute significantly to the survival rate of the infant.

Thus, it would be advantageous to develop a device that is capable of providing parent-infant attachment functions such as voice and heartbeat to the preterm or full term infant or in the absence of biological parents during this crucial early development period.

### SUMMARY OF THE INVENTION

A stimulation and soothing device for an infant is disclosed. The device includes a pouch large enough to accommodate the lower torso of an infant when the infant is placed in a reposed position therein. A backing piece includes first and second wings that wrap over the infant's shoulder and arms and secure to the pouch front panel. Each wing includes a pocket in which an insertable and programmable device may be housed. The insertable and programmable devices are provided for infant stimulation and soothing and may include a sound transducer and/or a portable digital media player. Additional pockets are provided for a temperature probe and for a phototherapy blanket.

### DESCRIPTION OF THE DRAWINGS

FIG. 1 is a perspective view of the infant stimulation and soothing device of the present invention;

FIG. 2 is a cross-sectional view taken along line 2-2 of FIG. 1;

FIG. 3 is a planar view of the pouch component of the infant stimulation and soothing device of the present invention shown in the open position;

FIG. 4 is a planar view of the front surface of the back piece component of the infant stimulation and soothing device of the present invention;

FIG. 5 is a planar view of the back surface of the back piece component of the infant stimulation and soothing device of the present invention showing cut-outs arranged for placement of medical positioners therein;

FIG. 6 is a planar view of the infant stimulation and soothing device of the present invention including the pouch component shown in the open position;

FIG. 7 is a planar view of the infant stimulation and soothing device of the present invention with an infant in a supine face up position;

FIG. 8 is a planar view of the infant stimulation and soothing device of the present invention illustrating the manner for closing the pouch component over an infant;

FIG. 9 is a planar view of the infant stimulation and soothing device of the present invention illustrating the pouch component in the closed position;

FIG. 9A is a detail view of an encircled portion of FIG. 9;

FIG. 10 is a planar view of the infant stimulation and soothing device of the present invention illustrating loading of certain accessory devices into pockets located on the right wing;

FIG. 11 is a planar view of the infant stimulation and soothing device of the present invention illustrating the right wing in the folded position; and,

FIG. 12 is a planar view of the infant stimulation and soothing device of the present invention illustrating the left and right wings in the folded position and illustrating the manner for loading a certain accessory device into a pocket and for attaching the scent retaining device.

### DETAILED DESCRIPTION OF THE PREFERRED EMBODIMENT

Referring now to FIG. 1, wherein like numbers indicate like parts throughout the drawings, there is shown at 10 a preferred embodiment of the infant blanket of the present invention illustrated containing an infant 12 lying in the supine face-up position. The infant blanket 10 includes a pouch 14 having a front panel 18 joined to a rear panel 22 by any suitable means, e.g., a common seam 26 (FIG. 3). The pouch component 14 is provided with zippers 30 on either or both opposite sides to enable the front and rear panels 18, 22 to be placed in an open position (FIG. 3) to allow the infant to be received within the pouch 14. Referring now to FIGS. 2 and 8, once the infant 12 has been placed onto the rear panel 22 of the pouch 14 in the supine face-up position, the zippers 30 may be utilized to close the pouch 14 to contain the infant 12 therein. A protective flap (not shown) is sewn into the panels 18, 22 and fastens over the zippers 30 to protect the infant 12 from possible contact with the zipper pulley mechanism 30a (FIGS. 8 and 9). As best illustrated in FIGS. 2 and 7, when the pouch 14 is placed in the closed position, an opening 34 is formed that is of sufficient size to enable the infant's head, neck, shoulders, arms, and upper chest to extend outside of the pouch while the remainder of the infant's body, e.g., feet, legs, lower chest portion, and diaper, remains within the pouch 14. The pouch 14 may be sized relative to the size of the infant contained therein. The pouch 14 is arranged to fit snuggly yet comfortably around the infant's lower chest, legs and feet to act as a “sack” for the infant's legs. The legs should be relatively free within the pouch 14 to allow optimal movement.

As best shown in FIGS. 8 and 9, the front panel 18 of the pouch 14 may be provided with a decorative element 39, e.g., a bow. Also, as best shown in FIGS. 9 and 9A, the rear panel 22 may be provided with opposed flaps 36 on which suitable closure devices 37 may be located and arranged to cooperate with closure devices 38 located on the front panel 18 to enable securement of the front panel 18 to the rear panel 22 in the vicinity of the opening 34 to form a continuous collar through which the infant 12 may extend. The closure devices may include cooperating hook and loop (Velcro) fasteners, clips, buttons, secured pins, adhesives, fabric, etc. Alternatively, although not shown, the flaps 36 may be provided on the front panel 18, as opposed to the rear panel 22.

Referring to FIG. 2, the pouch 14 may also include a substantially transparent panel 46 attached by any suitable means, e.g., a stitch 47 or sewing, to the interior surface of the rear panel 22 forming a pouch pocket arranged for the receipt of a light-emitting phototherapy or UV Bilirubin blanket 50 therein for the treatment of jaundice or hyperbilirubinemia. The pouch 14 may be made of any material or fabric suitable for infant wear. Examples are not confined to, but may include cotton, polyester, fleece, or other skin compatible materials that are breathable and machine washable and provide temperature stability characteristics suitable for the care of a newborn.

As best shown in FIGS. 2, 4 and 5, a backing piece 54 includes a top layer 58 secured to a bottom layer 62 by any suitable means, e.g., stitches 64 (FIG. 2). The top layer 58 of the backing piece 54 is arranged for securement to the rear panel 22 of the pouch 14 by any suitable fastening mechanism, e.g., cooperating hook and loop fasteners 55, located on the backing piece top layer 58 and the pouch rear panel 22. The backing piece 54 is provided with sufficient cushioning material to support the neck and head of the infant to minimize the occurrence of positional plagiocephaly, or flat head syndrome, which may develop from pressure against an infant's soft and malleable skull. The incidence of plagiocephaly has dramatically increased since 1992 when, to reduce the risk of SIDS (Sudden Infant Death Syndrome), parents and medical professionals were instructed to always place the infant in the supine (back) position to sleep.

The backing piece 54 includes left and right wings 66, 70. Each wing 66, 70 is arranged for wrapping from an unfolded position (FIGS. 4 through 7) to a folded position (FIGS. 1, 11 and 12) wherein the wing extends over the infant's shoulder, arms and torso. The wings 66 and 70 may include a scalloped edge 66a and 70a to provide a decorative appearance of angel wings. Referring to FIGS. 2 and 11, when in the folded position, the right wing 70 is arranged for attachment to the outside surface of the pouch 14, utilizing any suitable fastening mechanism 78, such as a Velcro fastener. Likewise, when in the folded position, the left wing 66 is arranged for attachment to the right wing 70 utilizing any suitable cooperating fastening mechanism 78, such as a Velcro fastener or snaps.

Referring now to FIGS. 2, 6 and 7, a pocket 80 is secured by any suitable means, e.g., hook and loop fasteners or sewing, to the top layer 58 of the right wing 70. An opening at the side of the pocket 80 may be self-closing, or closed by conventional means such as by mating Velcro strips, mating snaps, etc. The pocket 80 is arranged for receiving a heartbeat simulator 86 (FIG. 10) in the form of a sound transducer that produces vibration and sound similar to a mother's (or father's) heartbeat which is sensed and heard by the infant 12. The housing of the heartbeat simulator 86 includes a sufficiently thin profile so that it may fit within the pocket 80. The pocket 80 is located on the right wing 70 such that when the right wing 70 is placed in the folded position, the heartbeat simulator 86 will contact the chest of the infant 12 through the pocket 80. It should be understood that as the infant 12 grows, the location of the pocket 80 on the right wing 70 may be readily adjusted so that the heartbeat simulator 86 continues to make contact with the appropriate position on the chest of the infant 12 when placed within the infant blanket 10.

The heartbeat simulator 86 will induce the infant 12 with feelings of contentment, warmth and comfort, and aesthetic satisfaction similar to those the infant 12 would experience if in contact with the mother. The heartbeat simulator 86 may include a prerecording of the mother's own heartbeat during a particular week of the mother's pregnancy, such as during a prenatal visit. Alternatively, the expecting mother's heartbeat could be recorded at 20 weeks, which is approximately the point in time when the infant in utero begins to respond to auditory stimulation.

The recording could be done in advance by the mother using a stethoscope with the ear piece removed from the acoustical tube to fit around a microphone coupled to a high fidelity recording circuit. The heartbeat simulator 86 will reassure the infant by producing a calming and soothing effect, thus inciting the infant 12 to go back to sleep. The volume of the heartbeat simulator 86 may be adjusted to be louder or softer within a range of intensity determined to be suitable and safe to an infant based upon data from in utero sound recordings to accurately simulate the mother's heartbeat.

A second pocket 90 may be secured by conventional means, e.g., hook and loop fasteners or by sewing, to any suitable location such as the top layer of the right wing 70 at a location that is in proximity to the pocket 80. An opening at the side of the pocket 90 may be self-closing, or closed by conventional means such as by mating Velcro strips, mating snaps, etc. The second pocket 90 may be utilized for receiving a digital temperature probe 94 (FIG. 2) for measuring and monitoring the temperature of the infant 12 while situated within the infant blanket 10 of the present invention. When the right wing 70 is placed in the folded position (FIG. 11), the temperature probe 94 situated within the pocket 90 will be in the vicinity of the infant's liver for obtaining an accurate body temperature. The temperature probe may be a disposable liquid crystal thermometer. It should be understood that as the infant grows, the location of the second pocket may be readily adjusted so that the temperature probe 94 remains within the vicinity of the infant's liver.

Referring now to FIGS. 5 and 12, a third pocket 98 may be secured by conventional means, such as by mating Velcro strips or snaps, to the bottom layer of the left wing 66. An opening at the side of the pocket 98 may be self-closing, or closed by conventional means such as by mating Velcro strips, mating snaps, etc. The third pocket 90 may be utilized for receiving a battery-operated and portable digital media player 102 (FIG. 12) including an external speaker located on the housing of the player 102 for recording and playing back sounds. The digital media player 102 includes a housing having a sufficiently thin profile so that it fits within the third pocket 98. For example, the digital media player 102 could be arranged for recording and playing back appropriate content. The content could be a parental voice to calm and sooth the infant with a familiar voice. For example, the parental voice may be the mother's voice or the father's voice reading from a popular children's book. Alternatively, the player 102 could be programmed to play a melody or sounds similar to those that would be audible to the infant 12 in the womb, such as digestive sounds for the same calming and soothing effect on the infant 12 in the absence of biological parents. Optionally, actual sounds could be recorded from the infant's mother and replayed through the digital media player 102. In an alternative embodiment, the player 102 could be programmed to play a prerecorded program of choice, e.g., choral or vocal music, voices in multiple languages, etc. The content may be produced by the mother or alternatively, provided by a vendor.

In this embodiment, the digital media player 102 comprises a portable MP3 player, such as an iPod Shuffle® audio player, manufactured by Apple Computer, Inc. Of course, any other suitable audio device could be used, e.g., an MP4 player or a WMV player. The digital media player 102 could be arranged to play at a predetermined time, for a predetermined interval, and at a predetermined range of volume that is considered safe for the infant 12, e.g., 50-65 dBa. The digital media player 102 may include a security feature requiring the user, e.g., a mother or caretaker, to enter an alphanumeric log-in and password as a prerequisite for obtaining access to operation of the digital media player 102. In this manner, the chance of unauthorized playback and/or overwriting of inappropriate content on the digital media player 102 may be greatly reduced. Optionally, the digital media player 102 could be plugged into a charging dock (not shown) located in proximity to the infant 12, the charging dock including an external speaker for playing pre-recorded content.

Through the use of existing technology, the digital media player 102 and/or the heartbeat simulator 86 may be programmed to turn on, turn off, and to adjust in volume and intensity in accordance with the infant's sleep cycle. Generally, it is understood that there are three distinct stages in the sleep-wake cycle of an infant: (1) awake, during which large body movements of the infant are detected, (2) active sleep, during which infants “twitch”, and (3) deep sleep, during which the infant moves very little at all. For example, referring now to FIG. 4, a pocket 105 may be secured by any suitable means, e.g., hook and loop fasteners or sewing, to the top layer 58 of the backing piece 54, the pocket 105 being arranged for receiving one or more motion detectors 104 for monitoring and measuring the physical motion of the infant's entire body during sleep and waking to determine the stage of infant's sleep-wake cycle. Examples of suitable motion detectors 104 include, but are not limited to small electromechanical motors such as micro electrical mechanical systems (MEMS). In the event a MEMS device or multiple MEMS devices are employed as motion detectors, due to their small size, the MEMS device(s) may be housed within a larger flexible plastic container or membrane suitably sized for slipping into and out of the pocket 105.

The motion detector 104 may be arranged to measure motion of the infant 12 and the rate of change of motion. The motion detector 104 is provided with an integrated signal transmitter, e.g., a radio frequency (RF) transmitter, and may interface in known ways with a receiver located on the digital media player 102 and/or on the heartbeat simulator 86. The motion detector 104 may transmit signals relating to motion patterns of the infant to the digital media player 102 and/or the heartbeat simulator 86 at predetermined transmission intervals. In this manner, based upon movement of the infant, operation of the digital media player 102 and the heartbeat simulator 86 may be adjusted to different levels of volume and intensity during the infant's sleep wake cycle.

For example, when the motion detector 104 detects that the infant is awake, it may send signals to the receiver located on the digital media player 102 causing the digital media player 102 to adjust to play content, e.g., the mother's voice, at full intensity within a predetermined range considered safe for an infant. The frequency and duration of periods for playing content could vary based on many factors, including the specific needs of the infant and as well as the infant's age. For example the playback could be arranged to play a predetermined soundtrack in a crescendo-decrescendo arrangement for a reasonable interval, e.g., between 10 and 30 minutes. Likewise, as the motion detector 104 detects the infant transitioning from awake to active sleep, i.e., from squirming to twitching movements, it may send a signal to the receiver located on the digital media player 102 causing the player 102 to adjust play content at a lower volume at similar reasonable intervals. As the motion detector 104 detects the infant transitioning from active sleep to deep sleep, i.e., from twitching movements to virtually no movement, it may send signals to the receiver on the digital media player 102 causing the player 102 to turn the volume off altogether. As the motion detector 104 detects the infant transitioning from deep sleep to awake, it may send signals to the receiver on the digital media player 102 causing the player 102 to gradually return play of content to the full intensity discussed above. Optionally, when the motion detector 104 detects the infant transitioning from deep sleep to awake, through an audible and/or visual indicator, the motion detector 104 may notify the caretaker that the infant is awake again.

Likewise, during a period when the heartbeat simulator 86 is activated, upon the motion detector 104 detecting that the infant is awake, it may send signals to the receiver on the heartbeat simulator 86 causing the simulator 86 to adjust its vibration intensity to a maximum level. When the motion detector 104 detects that the infant has transitioned from awake to active sleep, or from active sleep to deep sleep, it may send signals to the receiver on the heartbeat simulator 86 causing it to diminish its vibration intensity to a suitably lower level.

The pockets 80, 90, and 98 and the panel 46 are provided to enable easy insertion and removal of the accessory devices 86, 94, and 102 so that the pouch 14 and backing piece 54 may be easily laundered. Moreover, it should be understood that there is no limit to the manner in which the pockets are attached to the wings. For example, the pockets could be attached by cooperating hook and loop fasteners, or Velcro, so that as the infant grows, the pockets are mobile and can be readily repositioned to new locations on the wings.

Referring now to FIGS. 1, 2 and 12, when the wings 66 and 70 are placed in the folded position, a device 106 retaining the scent of the mother may be secured by conventional means, such as by mating Velcro strips 110, to either wing in proximity to the infant's head. In these figures, the device 106 is shown attached to the right wing 70. The scent retaining device 106 is composed of a supple fabric such as cotton, silk or manmade blends that will absorb a portion of the mother's scent. The scent retaining device 106 is soft to the touch and nonirritating to the skin thus allowing the mother to wear the scent retaining device 106 underneath her undergarments and in contact with her skin for prolonged periods of time of twelve to twenty-four hours or more to absorb a portion of the mother's scent that can thereafter be detected by the infant when the device 106 is attached to either wing 66, 70. In this manner, the scent retaining device 106 will stimulate the infant's olfactory senses. It should be understood that although the figures illustrate the scent retaining device 106 as being heart-shaped, other shapes may be employed without departing from the invention.

Referring now to FIGS. 2 and 5, the infant blanket 10 of the present invention is provided with an infant medical positioner comprising two sets of medical positioners (or noodles) to limit movement of the infant 12 and provide physiologic positioning (in either mild extension or flexion) of the neck, shoulder girdle, thoracic and lumbar spine, and hips. A first set of medical positioners 114, 118, 122, and 126, arranged in a predetermined pattern, e.g., a tic-tac-toe pattern, are situated between the top layer 58 and the bottom layer 62 of the backing piece 54. The first set of medical positioners is positioned on the backing piece 54 at a location suitable for providing support to the shoulders of the infant 12. A second set of medical positioners 132, 134, 138, and 142 is arranged in a similar pattern and are positioned within the backing piece 54 to provide support to the pelvis area of the infant 12. It should be understood that the medical positioners are repositionable to provide support to different areas of the infant's anatomy or as the infant grows. The medical positioners, in combination with the wings and the pouch, conform to the infant 12 and place the infant in a physiologically safe and appropriate position for sleep. Each medical positioner is formed of a resilient foam material having a density for supporting the infant 12. Although the figures illustrate the medical positioners as being rectangular in cross-section, other cross-sectional shapes, e.g., circular, semi-circular, or triangular, may be utilized in accordance with the invention.

Referring now to FIGS. 6-10, in FIG. 6, the pouch 14 is shown attached to the backing piece 54 in the manner described above and in the open position to enable placement of the infant 12 within the pouch 14. As best shown in FIGS. 7 and 8, once the infant 12 is placed onto the pouch rear panel 22, the pouch 14 may be closed by pulling the zipper pulley mechanisms 30a located on each side of the pouch 14 in the direction indicated by arrows 31a such that the infant's head, neck, shoulders, arms and upper chest extend outside the pouch 14 while the remainder of the infant's body is positioned within the pouch. Thereafter, as best shown in FIGS. 9 and 9a, the opposed flaps 36 may be utilized to enable securement of the front panel 18 to the rear panel 22 in the vicinity of the opening 34 to form a continuous collar. As best shown in FIG. 10, once the pouch 14 has been closed around the infant 12, the accessory devices, e.g., the heartbeat simulator 86 and the temperature probe 94, may be inserted into their respective pockets 80 and 90. Thereafter, as best shown in FIG. 11, the right wing 70 may be folded over and secured to the pouch 14 utilizing suitable closure devices, e.g., cooperating hook and loop (Velcro) fasteners. Referring now to FIG. 12, the left wing 66 may be folded over and secured to the right wing 70 utilizing similar closure devices. Once in the folded position, the digital media player 102 may be inserted into its pocket 98 and the scent retaining device 106 may be attached to the outside surface of the folded right wing 70. With the wings 66 and 70 folded over the infant's shoulders, the combination of elements described above provides an infant stimulation and soothing device that conforms to the infant 12 and places the infant in a physiologically safe and appropriate position for sleep.

I claim:

1. An infant blanket with insertable and programmable devices to provide stimulation and soothing to an infant comprising:
a. a pouch large enough to accommodate at least the lower torso of an infant when the infant is placed in a supine position therein, said pouch including an upper opening through which the head, arms and upper torso of the infant can extend, said pouch including front and rear panels, said panels being connected by a bottom edge;
b. a backing piece including a first wing and a second wing, each said wing arranged for wrapping from an unfolded position to a folded position wherein said wing extends over the infant's shoulder and arms, said backing piece arranged for attachment to said pouch;
c. at least one insertable and programmable device; and,
d. a first wing pocket situated on the inside surface of said first wing and a second wing pocket situated on the outside surface of said second wing, said first and second wing pockets arranged for housing said at least one insertable and programmable device therein.

2. The infant stimulation and soothing device of claim 1, wherein said pouch includes at least one open side edge that is closeable by a closure mechanism.

3. The infant stimulation and soothing device of claim 2, wherein said closure mechanism comprises at least one of: a snap fastener, a zipper, and a hook and loop fastener.

4. The infant stimulation and soothing device of claim 1, wherein said insertable and programmable device comprises a sound transducer arranged to emit vibrations as a perceptible effect.

5. The infant stimulation and soothing device of claim 4, wherein said insertable and programmable device comprises a portable digital media player including a speaker for emitting sound.

6. The infant stimulation and soothing device of claim 5, wherein said sound transducer is housed within said first wing pocket and said portable digital media player is housed within said second wing pocket.

7. The infant stimulation and soothing device of claim 6, wherein said sound transducer is arranged to simulate perceptible effects selected from the group consisting of a human heart, the heartbeat of the infant's mother, the heartbeat of the infant's mother during pregnancy, and the heartbeat of the infant's father.

8. The infant stimulation and soothing device of claim 1, additionally comprising an aromatic element configured to release a scent as the perceptible effect, said aromatic element arranged for securement to one of said first and second wings.

9. The infant stimulation and soothing device of claim 5, wherein said prerecorded sound is selected from the group consisting of the voice of the infant's birth mother, the voice of the infant's father, voice of a frequent care giver, instrumental music, songs, lullabies, cardiovascular sounds, digestive sounds, and phonetic presentations in various languages.

10. The infant stimulation and soothing device of claim 9, wherein said portable digital media player is selected from the group consisting of an MP3 player, an MP4 player, a WMV player, and an Apple iPod.

11. The infant stimulation and soothing device of claim 1, wherein said pouch is manufactured from materials consisting of cloth, fabric, and textile.

12. The infant stimulation and soothing device of claim 1, wherein said wings are manufactured from materials consisting of cloth, fabric, and textile.

13. The infant stimulation and soothing device of claim 1, additionally comprising a pocket located on said backing piece for housing a temperature probe.

14. The infant stimulation and soothing device of claim 1, wherein said pouch additionally comprises a pouch pocket formed of a transparent panel, said pouch pocket arranged for receipt of a phototherapy or UV Bilirubin blanket therein for treatment of jaundice.

15. The infant stimulation and soothing device of claim 1, additionally comprising a motion detector situated on said backing piece for detecting movement of the infant and controlling said at least one insertable and programmable device in response to the infant's sleep cycle.

16. The infant stimulation and soothing device of claim 15, wherein said motion detector is a MEMS sensor.
